(19) United States
(12) Patent Application Publication
(10) Pub. No.: US 2005/0185758 A1
(43) Pub. Date: Aug. 25, 2005

(54) Method for planning the radiation therapy for a patient

(76) Inventors: Herbert Bruder, Hoechstadt (DE); Thomas Flohr, Uehlfeld (DE); Christoph Panknin, Erlangen (DE)

Correspondence Address: HARNESS, DICKEY & PIERCE, P.L.C., P.O.BOX 8910, RESTON, VA, 20195, US

(21) Appl. No.: 11/053,879

(22) Filed: Feb. 10, 2005

(30) Foreign Application Priority Data
Feb. 10, 2004 (DE) 10 2004 006 548.9

(51) Int. Cl. A61N 5/10

(52) U.S. Cl. 378/65

(57) ABSTRACT

A method is for producing CT scans, particularly for planning radiation therapy for a patient in the chest region, where the exclusive use of CT data is used to infer the respective motion phase of a scanned region. This information is used to produce phase-specific CT images. These images are used for the irradiation planning in the chest region, in particular.

The present application hereby claims priority under 35 U.S.C. §119 on German patent application number DE 10 2004 006 548.9 filed Feb. 10, 2004, the entire contents of which are hereby incorporated herein by reference.

### FIELD OF THE INVENTION

The invention generally relates to a method for producing CT scans. Particularly it relates to a method for planning radiation therapy for a patient in the chest region, where optimized irradiation of an unhealthy region, usually of a tumor, is performed by determining the position and extent of this region and of the patient using computed tomographic scans. Subsequently these position data are used to choose an irradiation distribution which produces the most effective possible specific dose load for the unhealthy region and the lowest possible specific dose load for the rest of the patient's tissue.

### BACKGROUND OF THE INVENTION

Irradiation planned in radiation therapy relates generally to the optimization of dose distribution in time and space in order to stipulate a method of radiation therapy which is intended to achieve a particular clinical effect, usually the elimination of a tumor. The aim of irradiation planning is to put a homogeneous dose of radiation into a target volume and at the same time to keep the dose in the surrounding normal tissue as minimal as possible. The target volume will generally be a tumor, possibly surrounded by a corresponding safety region. The volume which is intended to be irradiated is in this case defined by clinical examinations, such as computed tomography. For the method of calculation in irradiation planning on the basis of knowledge of the geometrical stipulations, reference is made to the book “Bestrahlungsplanung” [Irradiation planning], Thieme-Verlag, ISBN 3137850029, the entire contents of which is hereby incorporated herein by reference.

One problem with irradiation planning, particularly with irradiation operations in the chest region, is that the patient is breathing for the duration of the irradiation, which may be several tens of minutes. This causes cyclic alteration of the position of the region which is to be irradiated. To date, such cyclic alterations in the position of a target volume during chest irradiation operations have not been taken into account in the irradiation planning, which results in uncertainty in the irradiation planning. In particular, another reason for the existence of this problem is that it is difficult to find a correlated signal for the motion of the lung or of the chest region which (signal) is correlated to the motion of the chest in a similar manner to an ECG, which is accompanied by the motion of the heart.

### SUMMARY OF THE INVENTION

It is therefore an object of an embodiment of the invention to find a method for irradiation planning in which the motion of the volume which is to be irradiated in the chest region is taken into account during the irradiation.

The inventors have discovered that it is possible to apply features of an inherently known method for detecting heart motions, solely from computed tomographic raw data, to the motion of the chest as well. Thereby, CT image data can be obtained in which individual specific motion phases of the entire motion cycle of the chest are extracted.

As a result, it is possible to display motion maxima for tumors situated in the chest, for example. Thus, in the irradiation planning, specifically these volumes which contain a tumor during the irradiation are intensively irradiated, while other healthy regions are deliberately omitted. This allows a significant reduction in the previously used “safety region” around the tumor, which is also included in the intensive irradiation during radiation treatment to date in order to achieve good prospects of success for destroying the tumor tissue. In this way, a smaller amount of healthy tissue is affected by the radiation treatment.

Accordingly, the inventors propose improving the inherently known method for producing CT scans, particularly for planning radiation therapy for a patient in the chest region, in which optimized irradiation of an unhealthy region, usually of a tumor, is performed. This can be done in one embodiment, for example, by determining the position and extent of this region and of the patient using computed tomographic scans and by subsequently using these position data to choose an irradiation distribution which produces the most effective possible specific dose load for the unhealthy region and the lowest possible specific dose load for the rest of the patient's tissue, by virtue of the computed tomographic scans being produced on the lung, which moves cyclically through breathing, with motion information being taken from imaging projection data from the CT itself and at least two different cycle phases being displayed. When the irradiation planning is carried out, a positional shift in the unhealthy region as a result of the cyclic motion can then be taken into account.

This inventive method of one embodiment allows very exact determination of at least the extreme areas of motion of the volume which is to be irradiated. Thus, the irradiation planning now has just a very small amount of uncertainty about the actual location of the target volume on account of the motion of the chest through breathing. As a result of this, fewer uncertainties are accepted and the “safety region” can be reduced during the irradiation planning.

Preferably, the motion information from the CT data is detected by forming a two-dimensional location integral for the weakening coefficients.

It may also be advantageous if the motion information from the CT data is detected by virtue of projection-by-projection and row-by-row summation of direct projection data taking place.

Also, the motion information from the CT data can be detected by virtue of projection-by-projection and row-by-row summation of direct projection data, preferably in the row direction or channel direction of a multirow detector used, taking place.

In the case of the variants mentioned last, the motion information is thus drawn just from the direct projection data, that is to say without using complementary projection data. In a development for this, however, it is also possible to determine motion information from the difference values for direct projection data and complementary projection data. The complementary projection data are to be understood to mean the data which are recorded with a 180° offset from the direct projection data and which thus—in the case of a static object—differ only in the contrary direction of radiation. If there is motion by an object, however, then a difference arises in the ascertained weakening coefficients depending on the direction of radiation.

Accordingly, the inventors also propose an embodiment including detecting the motion information from the CT data by performing projection-by-projection and row-by-row difference value determination between direct projection data and complementary projection data. Preferably, subsequent projection-by-projection and row-by-row summation of the difference values, preferably in the channel direction of a multirow detector used, can take place. In this context, prior to the calculation it is possible for projection-by-projection interpolation of multirow direct projection data and of complementary projection data onto a common z position to take place.

Advantageously, the motion information from the CT data can also be mapped as a global motion function over the scanning time, with bandpass filtering to suppress possible parasitic frequencies (which may be caused by the gantry rotation or other artifacts) being applied to the global motion function f appropriate.

In line with an embodiment of the invention, a CT image of a particular cycle phase can also be produced by using data from at least two cycles and from the same cycle phase. As a result, it is possible to increase the image quality, since the exposure times or the length of the observed cycle phases can become correspondingly shorter by virtue of the full 180° data collection being distributed over a plurality of cycles, preferably two cycles. In principle, such methods for data collection for CT scans are known from ECG-gated cardio scans, for example, but in this case a secondary source is not used for the motion information, but rather the motion information is taken from the CT data themselves.

It is also within the scope of an embodiment of the invention if, instead of a few scans of the motion maxima, CT images from a multiplicity of cycle phases, that is to say a 3D image series or 3D film, are calculated over the motion cycle.

It is furthermore within the scope of an embodiment of the invention if, on the one hand, the data from particular cycle phases from a plurality of motion cycles are compiled prior to the calculation of incomplete CT image stacks, or, on the other hand, the data from particular cycle phases from a plurality of motion cycles are compiled after the calculation of incomplete CT image stacks. By way of example, these latter variants may also be implemented in connection with cardio CT scans.

It will also be pointed out that aspects of the inventive method can be carried out by performing the CT either with a focus which makes a circular motion or with one which makes a spiral motion around the patient. In addition, parallel sorting of the measurement data can take place prior to performance of the calculation for detecting the motion from the CT data.

### BRIEF DESCRIPTION OF THE DRAWINGS

The invention is described in more detail below using a preferred exemplary embodiment with reference to FIGS. 1 to 7, the following reference symbols being used in the figures: 1: computed tomogram; 2: X-ray tube; 3: multirow detector; 4: patient's table; 5: system axis/z axis; 6: gantry; 7: patients; 8: control and processing unit; 9: control and data line; 10: radiation therapy appliance; 11: gantry; 12: patient's table; 13: axis of rotation of the radiation source; 14: control and processing unit; 15.1: lung; 15.2: lung; 16: heart; 17: spinal column; 18.x: direct X-rays; 18.x′: complementary X-ray; 19: circle; 20: curve of the motion function; P₁-Pₙ: programs; Prg₁-Prgₙ: programs.

In the figures, specifically:

FIG. 1 shows a computed tomogram;

FIG. 2 shows a radiation therapy appliance;

FIG. 3 shows a representation for direct projection data;

FIG. 4 shows an unfiltered motion function from the CT data;

FIG. 5 shows a spectral distribution for the motion function;

FIG. 6 shows a filtered motion function with marked function maxima;

FIG. 7 shows a representation for direct and complementary projection data for difference value formation.

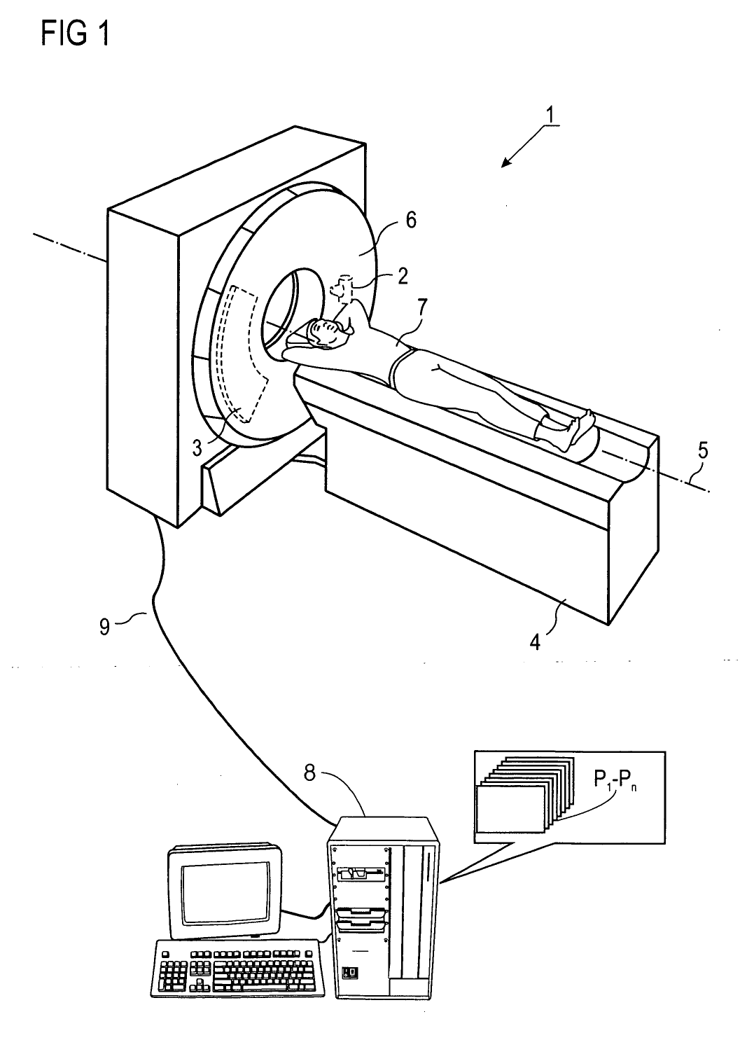

In one preferred variant of the inventive method, the inventors propose planning the irradiation for a tumor in the chest region by determining the position data for the tumor using a CT, with the CT scan being able to be produced, by way of example, using a CT in line with the schematic illustration in FIG. 1.

### DETAILED DESCRIPTION OF THE EXEMPLARY EMBODIMENTS

FIG. 1 shows such a computed tomogram 1, which, for the purpose of scanning a patient, has an X-ray tube 2, rotating on a gantry 6 around a patient 7, with a concomitantly rotating multirow detector 3 arranged opposite. For the purpose of scanning, the patient 7 is pushed using a patient's table 4, which can be moved along the system axis (z axis) 5, while the gantry 6 is rotating, which means that a spiral scan is produced relative to the patient 7. This operation is controlled by a control and processing unit 8 which is connected by way of control and data lines 9 to the drive unit of the CT, to the X-ray tube, to the drive for the patient's table and to the multirow detector 3. This control and processing unit 8 also evaluates the collected CT data and calculates the CT images. This is essentially also done using the programs P₁-Pₙ (shown symbolically).

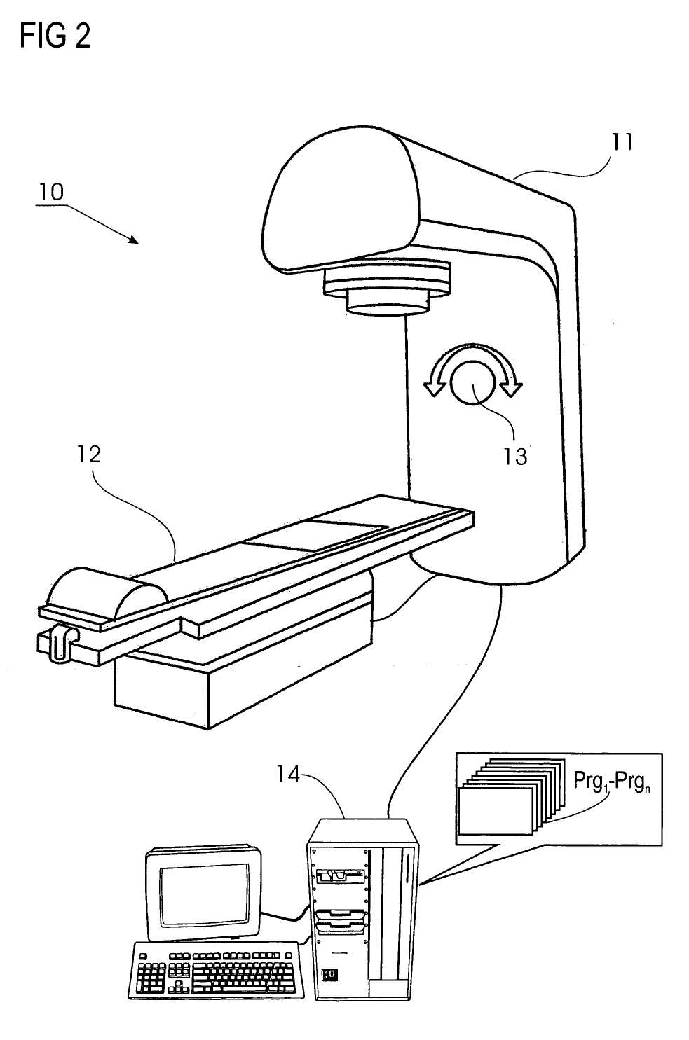

With appropriate configuration, this processing unit may also be used for the irradiation planning after the target volume or tumor has been located, but dedicated computers into which the CT images have been transmitted digitally beforehand are usually used for this purpose. When the irradiation planning has been performed, the therapeutic irradiation of the patient is carried out using a radiation therapy appliance, which is shown by way of example in FIG. 2. Such a radiation therapy appliance 10 includes a variably adjustable radiation source, in this case shown as a linear accelerator with beam deflection, which rotates about an axis 13 around a patient (who is situated on a movable patient's table 12) in the gantry 11 under the control of a control and processing unit 14.

Normally, the irradiation is controlled in this context using software, which is represented by way of example by the programs Prg₁-Prgₙ. In principle, the processing unit which is available here may also be used for the irradiation planning, in which case the CT image data need to be transmitted to it. It goes without saying that when the patient is in position it is necessary to ensure that the position data for the tumor are transmitted from the CT to the radiation therapy appliance correctly.

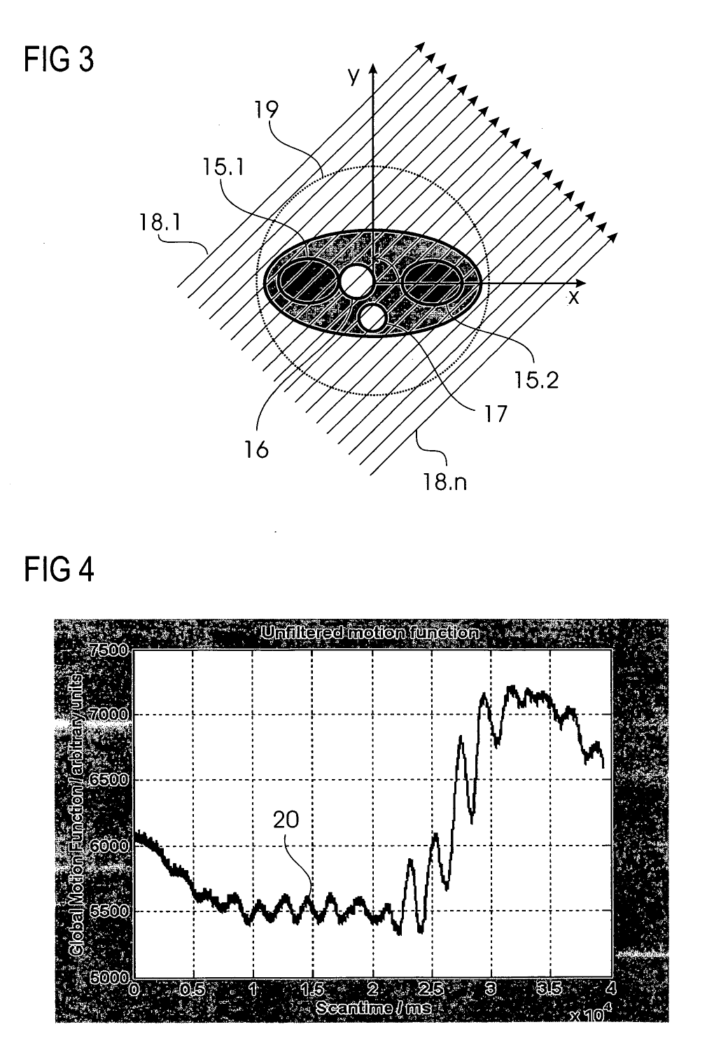

In line with an embodiment of the invention, to show and define the position of the tumor, the patient is scanned with X-rays using a CT, as shown schematically in FIG. 3. This figure shows the cross section of a patient 7 with the lungs 15.1 and 15.2, including the heart 16 and the spinal column 17. The X-rays 18.x are shown parallel, as they appear after—only optional—parallel rebinning.

According to an inventive concept of one embodiment, the CT data are now used to produce a motion function which records the sum of the weakening coefficients for an area, for example the circle 19. Expressed as a formula, this gives the following integral, for example:
I^{global}(θ(t))=∫dp·h(θ(t),p,q))

Here, I^{global}(θ(t)) describes the motion function over the angle of rotation θ, p describes the parallel coordinate, h describes the projection data and q describes the row number.

In principle, it is also possible to perform additional summing over the detector rows z, resulting in the following:

$${I}^{global} ( θ ( t ) ) = {\sum }_{q} \int ⅆ p \cdot h ( θ ( t ) , p , q ) )$$

When the motion function is plotted over the scan time t or the angle of rotation θ which is proportional thereto, both variants produce a curve 20, as shown in FIG. 4. In a first approximation, it would actually be possible to assume that the integral shown does not alter over the weakening coefficients despite the patient's breathing motions and the associated expansion of the chest, since the area covered by the integral should not experience any alteration of mass as a result of the breathing motion. Nevertheless, oscillations which are in the range of the breathing frequency can be seen in the motion function. Upon closer examination of the breathing motion, however, it can be seen that the breathing motion also causes mass displacements in the z direction and as a result there is also an alteration in the summed weakening coefficients.

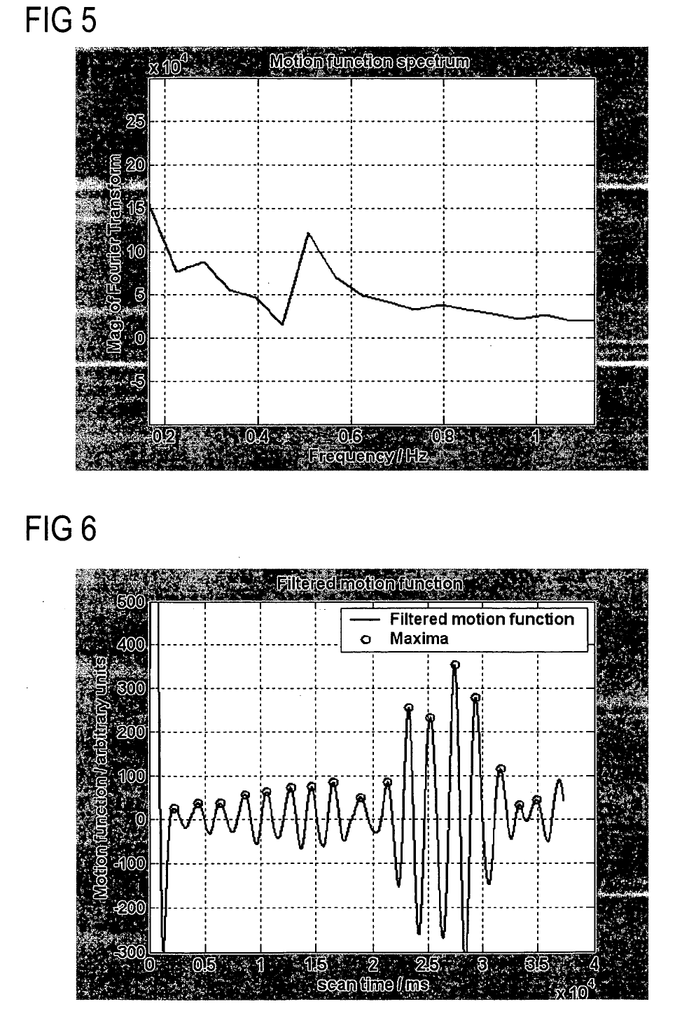

FIG. 5 shows the result of a Fourier analysis from the motion function in FIG. 4, with the magnitude of the Fourier transform being plotted against frequency. In this case, it is possible to see a distinct rise, particularly in the range of the breathing frequency at approximately 0.5 Hz.

To illustrate these frequencies more clearly, it is possible to perform bandpass filtering over the motion function, the result of which is shown in FIG. 6. In this case, it can be seen that clear maxima and minima are discernible in the filtered motion function, these being correlated to the breathing motion.

In line with an embodiment of the invention, this resultant motion information can be used to obtain CT scans of particular motion phases in the chest, as is usual with cardio scans, for example. At the same time, these phase-dependent CT images of the chest can be used to define an area of motion which contains a tumor during a breathing motion, and hence more accurate irradiation planning than previously can take place.

In line with an inventive concept of an embodiment, the motion function and CT image data phase-selected using the motion frequency which is discernible from the motion function can be obtained and hence image material can be collected over one or more motion cycles and processed on a phase-selected basis.

However, adding to the method outlined above, it is also possible to use the direct differences between rays which are in opposite directions but at the same location (and which may also be interpolated if appropriate) for the purpose of motion detection, instead of considering direct projection data.

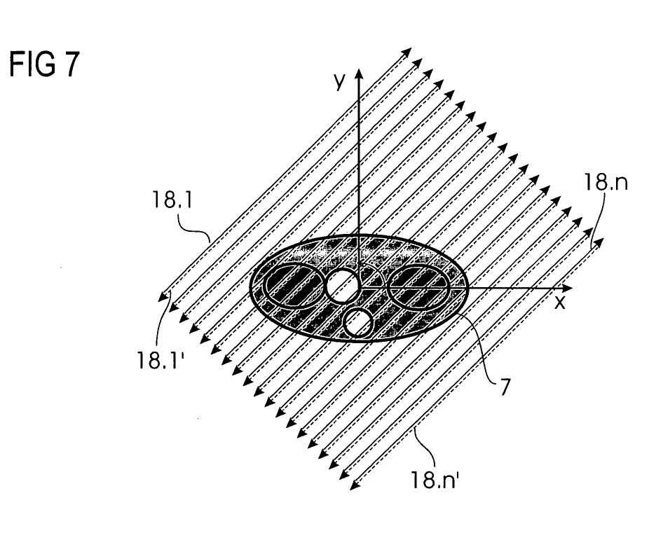

For the purposes of illustration, FIG. 7 shows—after prior optional parallel rebinning—direct rays 18.x (which are shown as solid lines) and complementary rays 18.x′ (which are shown as dashed lines) upon passing through a patient 7. With regard to the representation of the rays, it should also be noted that the direct and complementary rays actually run congruently and are shown with a slight offset only to improve discernability.

If the rays are recorded simultaneously, then both rays show the same overall weakening. If the rays are recorded with a slight timing offset and the patient has not moved in the meantime, then there is no resultant change. However, if the patient has moved between the recording times for the rays in opposite directions, there is a resultant difference in the weakening of the rays in opposite directions. This difference can be used in order to detect a patient's chest motion and, on the basis of the detected motion cycle, to produce gated CT images which can be used especially during irradiation planning in order to determine the areas of motion of tumors more accurately and to include them in the dose planning.

In line with this outlined variant, the inventive method may have the following steps, for example:
acquisition of a multirow CT spiral data record of the chest volume using a suitable table feed unit;
optional row-by-row parallel sorting of the measurement data;
projection-by-projection interpolation of the multirow, direct projection data h(θ,p,z_{q}) and of the 180⁰-offset complementary projection data h(θ(t+Tᵣₒₜ/2),p,z^{π}_{q}) onto a common z position z*q with θ: angle of projection, p: parallel coordinate, q: row number, z_{g}: z position of the detector center in the angle of projection θ, z^{π}_{q}: z position of the detector center in the angle of projection θ(t+Tᵣₒₜ/2), Trot: gantry rotation time, and t: acquisition time
projection-by-projection and row-by-row determination of the difference between direct rays and the 180⁰-offset complementary rays h(θ(t+Tᵣₒₜ/2), −p, z*_{q})·h (θ, p, z*_{q})
projection-by-projection and row-by-row summation of the difference value signal in the channel direction (p direction) S(θ(t),z*_{q});
projection-by-projection summation in the row direction (optional) (q direction) S(θ(t),z*_{q})′;
bandpass filtering of S(θ(t))′ in order to suppress parasitic frequencies which are caused by the gantry rotation, with the result of a reconstructed motion curve S(θ(t))″)
phase-correlated reconstruction of CT raw data, for which partial rotation data should be used to obtain the best possible time resolution; suitable reconstruction algorithms are published in T. Flohr, B. Ohnesorge, “Heart-Rate Adaptive Optimization of Spatial and Temporal Resolution for ECG-Gated Multi-slice Spiral CT of the Heart”, JCAT vol. 25 No. 6, 2001 and in H. Bruder, K. Stierstorfer, B. Ohnesorge, S. Schaller, T Flohr; “A Novel Rekonstruktion Scheme for Cardiac Volume Imaging with MSCT Providing Cone Correction”, SPIE Med. Imag. Conf., vol. 4684, pp. 60-71, 2002, for example. The entire contents of each of these publications is hereby incorporated herein, in full, by reference.
the phase selection can be made in the reconstructed motion curve S(θ(t))″″; followed by
correct-phase volume representation of the tumor region in order to determine the maximum tumor motion or the range of motion of the tumor.

All in all, an embodiment of the invention thus provides a way of inferring the respective motion phase of a scanned region through the exclusive use of CT data and of using this information to produce phase-specific CT images, these images being able to be used, in particular, for irradiation planning in the chest region.

It goes without saying that the aforementioned features of the invention can be used not only in the respective indicated combination but also in other combinations or on their own without departing from the scope of the invention.

Any of the aforementioned methods may be embodied in the form of a system or device, including, but not limited to, any of the structure for performing the methodology illustrated in the drawings.

Further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable involatile memories, such as ROMs and flash memories, and hard disks. Examples of the removable medium include, but are not limited to, optical storage media such as CD-ROMs and DVDS; magneto-optical storage media, such as MOs; magnetism storage media, such as floppy disks (trademark), cassette tapes, and removable hard disks; media with a built-in rewriteable involatile memory, such as memory cards; and media with a built-in ROM, such as ROM cassettes.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for producing computed tomographic scans for planning radiation therapy for a patient in an unhealthy region, the method comprising:
determining a position and extent of the region and of the patient using computed tomographic scans;
choosing, using produced position data, an irradiation distribution which produces a most effective possible specific dose load for the unhealthy region and a lowest possible specific dose load for a remainder of the patient's tissue, wherein the computed tomographic scans are produced on a lung of the patient moving cyclically through breathing, with motion information being taken from imaging projection data from the computed tomographic scans; and
selectively displaying at least two different cycle phases.

2. The method as claimed in claim 1, wherein the irradiation planning takes into account a positional shift in the unhealthy region as a result of the cyclic motion.

3. The method as claimed in claim 1, wherein the motion information from the CT data is detected by forming a two-dimensional location integral for the weakening coefficients.

4. The method as claimed in claim 1, wherein the motion information from the CT data is detected by virtue of projection-by-projection and row-by-row summation of direct projection data taking place.

5. The method as claimed in claim 1, wherein the motion information from the CT data is detected by virtue of projection-by-projection and row-by-row summation of direct projection data.

6. The method as claimed in claim 1, wherein the motion information from the CT data is detected by virtue of projection-by-projection and row-by-row difference value determination between direct projection data and complementary projection data with subsequent projection-by-projection and row-by-row summation of the difference values.

7. The method as claimed in claim 6, wherein, prior to the calculation, projection-by-projection interpolation of multirow direct projection data and of complementary projection data onto a common z position takes place.

8. The method as claimed in claim 1, wherein the motion information from the CT data is mapped as a global motion function over the scan time.

9. The method as claimed in claim 8, wherein bandpass filtering is applied to the global motion function.

10. The method as claimed in claim 1, wherein a CT image of a particular cycle phase is produced by using data from at least two cycles and from the same cycle phase.

11. The method as claimed in claim 1, wherein CT images of a multiplicity of cycle phases in the motion cycle are calculated.

12. The method as claimed in claim 1, wherein the data from particular cycle phases from a plurality of motion cycles are compiled prior to the calculation of incomplete CT image stacks.

13. The method as claimed in claim 1, wherein the data from particular cycle phases from a plurality of motion cycles are compiled after the calculation of incomplete CT image stacks.

14. The method as claimed in claim 1, wherein circular scanning takes place around the patient.

15. The method as claimed in claim 1, wherein spiral scanning takes place around the patient.

16. The method as claimed in claim 1, wherein parallel sorting of the measurement data takes place prior to the calculation.

17. A computed tomogram, comprising program means for implementing the method of claim 1.

18. The method as claimed in claim 2, wherein the motion information from the CT data is detected by forming a two-dimensional location integral for the weakening coefficients.

19. The method as claimed in claim 1, wherein the motion information from the CT data is detected by virtue of projection-by-projection and row-by-row summation of direct projection data in at least one of the row direction and channel direction of a multirow detector.

20. A computer program, adapted to carry out the method of claim 1, when run on a computer device.

21. A computer readable medium, including the computer program of claim 20.

22. A method, comprising:
determining a position and extent of an unhealthy region of a patient using computed tomographic scans;
choosing, using produced position data, an irradiation distribution which produces a relatively high effective dose load for the unhealthy region and a relatively low dose load for a remainder of the patient's tissue, wherein the computed tomographic scans are produced on a lung of the patient moving cyclically through breathing, with motion information being taken from imaging projection data from the computed tomographic scans; and
selectively displaying at least two different cycle phases.

23. A device, comprising program means for implementing the method of claim 1.

24. A computer program, adapted to carry out the method of claim 22, when run on a computer device.

25. A computer readable medium, including the computer program of claim 24.
